(19) United States
(12) Patent Application Publication
(10) Pub. No.: US 2017/0027543 A1
(43) Pub. Date: Feb. 2, 2017

(54) ULTRASONIC PROBE AND ULTRASONIC APPARATUS

(71) Applicant: Seiko Epson Corporation, Tokyo (JP)

(72) Inventor: Kanechika KIYOSE, Matsumoto (JP)

(21) Appl. No.: 15/209,935

(22) Filed: Jul. 14, 2016

(30) Foreign Application Priority Data
Jul. 29, 2015 (JP) 2015-149249

(51) Int. Cl. A61B 8/00; A61B 8/08; A61B 8/14

(52) U.S. Cl. CPC A61B 8/4455; A61B 8/145; A61B 8/4411; A61B 8/4494; A61B 8/5207; A61B 8/4254

(57) ABSTRACT

An ultrasonic probe includes a housing shaped like a thin plate and having a sensor surface as a transmitting/receiving surface for an ultrasonic wave, and a hand part having an axial direction, and connected to a side surface of the housing at one end in the axial direction, the side surface crossing the sensor surface of the housing. Thus, it is possible to pick up the hand part to dispose the ultrasonic probe to a desired position, and it becomes possible to check an internal tomographic image of the object viewed from an arbitrary direction without suspending the operation.

### BACKGROUND

1. Technical Field

The present invention relates to an ultrasonic probe, and an ultrasonic apparatus.

2. Related Art

In the past, there has been known an ultrasonic apparatus for imaging an internal cross-sectional structure of an object using an ultrasonic wave (see, e.g., JP-A-2005-28050 (Document 1)).

The ultrasonic probe described in Document 1 is an ultrasonic probe used when checking an internal tomographic image of, for example, an organ in a living body during an operation in the medical field. The ultrasonic probe has a probe main body having a vibrator part disposed in the tip part, the vibrator part being provided with an ultrasonic vibrator, and a cable for performing signal input/output is connected to an end of the probe main body, the end being located on the opposite side to the vibrator part. The cable extends toward the opposite side to the output direction of the ultrasonic wave from the vibrator part. In the ultrasonic probe having such a configuration, in the case in which a practitioner obtains an internal tomographic image of an operation object using the ultrasonic probe during, for example, the operation, it is possible to release the cable in a direction of getting away from the operation object when pressing the ultrasonic probe against the surface of the operation object.

Incidentally, in such an ultrasonic probe as described above, the ultrasonic probe is pressed against the operation object from above to check the internal tomographic image of the operation object. Therefore, in the case in which the practitioner checks the internal tomographic image of the operation object during the operation, it is necessary to suspend the operation to perform measurement by the ultrasonic probe and the check of the internal tomographic image, and then separate the ultrasonic probe from the operation object to a position where the ultrasonic probe does not interfere with the operation. Further, since the cable extends toward the opposite side to the output direction of the ultrasonic wave, it is unachievable to check the internal tomographic image from the side or the reverse of the organ to be the operation object.

### SUMMARY

An advantage of some aspects of the invention is to provide an ultrasonic probe and an ultrasonic apparatus each capable of checking the internal tomographic image of an object viewed from an arbitrary direction without suspending the operation to the object.

An ultrasonic probe according to an application example of the invention includes a housing shaped like a thin plate and having a transmitting/receiving surface for an ultrasonic wave, and a hand part having an axial direction, and connected to a side surface of the housing at one end in the axial direction, the side surface crossing the transmitting/receiving surface of the housing.

In this application example, the ultrasonic probe is provided with the hand part disposed on the side surface crossing the transmitting/receiving surface for the ultrasonic wave of the housing shaped like a thin plate.

In such a configuration, since the housing is shaped like a thin plate, and the hand part is disposed on the side surface of the housing, by holding the hand part to insert the housing to the side or the reverse of the object, the ultrasonic probe can be disposed at a desired position.

Thus, when performing the operation to, for example, an organ in the living body, in the case in which the practitioner checks the internal tomographic image of the measurement object such as the operation object during the operation, it becomes unnecessary to perform such a cumbersome operation that the operation is suspended to press the ultrasonic probe against the surface of the measurement object to check the internal tomographic image, and then move the ultrasonic probe away to a position where the ultrasonic probe does not hinder the operation to continue the operation. In other words, in the present application example, since the ultrasonic probe is disposed in the side or the reverse of the measurement object while holding the hand part, it is unnecessary to press the ultrasonic probe against the measurement object or to remove the ultrasonic probe from the measurement object during the operation, the operation process can be shortened, and a prompt operation becomes possible. Further, on this occasion, since the hand part is connected to the side surface of the housing having a thin plate shape, even if the housing is inserted to the side or the reverse of the measurement object, there is no chance for the hand part to injure the measurement object and other organs in the living body, and to hinder the operation. Moreover, since it is possible to press the transmitting/receiving surface of the ultrasonic probe against the measurement object at an arbitrary position using the hand part, it is possible to check the internal tomographic image of the measurement object from an arbitrary direction, and the operation high in efficiency becomes possible.

In the ultrasonic probe according to the application example, it is preferable that the hand part extends in a direction parallel to a surface direction of the transmitting/receiving surface.

In the application example with this configuration, the hand part extends in a direction parallel to the surface direction of the transmitting/receiving surface. In such a configuration, due to the hand part, the ultrasonic probe can easily be inserted in the desired position without the interference of the hand part with the operation object and so on. Further, in the case of disposing the ultrasonic probe to the reverse of the organ, and so on, if the hand part extends in a direction crossing the transmitting/receiving surface, there is a possibility that the hand part injures other parts and so on. However, in the present application example, the interference of the hand part with other parts can also be suppressed, and the ultrasonic probe can safely be disposed at the desired position.

In the ultrasonic probe according to the application example, it is preferable that the hand part is disposed so as to detachably be attached to the housing.

In the application example with this configuration, since the hand part is detachably attached to the housing, the hand part can be detached after disposing the ultrasonic probe at the desired position, and thus the operation is not hindered. Thus, the more efficient operation can be performed.

In the ultrasonic probe according to the application example, it is preferable that the hand part is provided with a first hand part connected to the housing, and a second hand part connected to the first hand part so that an angle with the first hand part can be changed.

In the application example with this configuration, the hand part is formed of the first hand part and the second hand part, and the second hand part is disposed so that the angle with the first hand part can be changed. Thus, it is possible to change the angle of the second hand part with the first hand part to move the second hand part to a position where the second hand part does not hinder the operation after holding the hand part to install the ultrasonic probe in the side or the reverse of the measurement object, and thus, there is no chance for the hand part to hinder the operation, and the efficient operation can be performed.

In the ultrasonic probe according to the application example, it is preferable that the hand part is provided with a cylindrical inner peripheral surface along the axial direction, and a cable to be connected to an ultrasonic sensor disposed inside the housing is disposed in a hollow part formed of the cylindrical inner peripheral surface.

In the application example with this configuration, the hand part has the cylindrical inner peripheral surface, and the cable to be connected to the ultrasonic sensor for performing the transmission/reception of the ultrasonic wave is disposed on the cylindrical inner peripheral surface. In such a configuration, it is not necessary to separately dispose the connection section of the cable to the housing, and the simplification of the configuration can be achieved.

In the ultrasonic probe according to the application example, it is preferable that the side surface of the housing is a convexly curved surface.

In the application example with this configuration, since the side surface of the housing is disposed to have the convexly curved surface, in the case of, for example, disposing the ultrasonic probe inside the living body, and so on, the problem that the organ in the living body is injured by a sharp angle part of the housing does not occur, and the ultrasonic probe can safely be disposed in the living body.

In the ultrasonic probe according to the application example, it is preferable that a thickness dimension along a normal line of the transmitting/receiving surface of the housing is no larger than 1 cm.

In the application example with this configuration, since the thickness dimension of the housing is no larger than 1 cm, when inserting the ultrasonic probe in the living body, there is no chance of exerting pressure on the inside of the living body, and the ultrasonic probe can safely be disposed.

In the ultrasonic probe according to the application example, it is preferable that the ultrasonic probe further includes a plurality of ultrasonic transducers each including a support film and a vibrator shaped like a film and adapted to vibrate the support film, and the ultrasonic transducers are arranged in an array along a surface direction of the transmitting/receiving surface.

In the application example with this configuration, there are provided the ultrasonic transducers each formed of the support film and the vibrator having a thin film shape, and the ultrasonic sensor is constituted by the ultrasonic transducers arranged in an array along the surface direction of the transmitting/receiving surface. In such a configuration, the height reduction becomes possible compared to the ultrasonic probe using, for example, a bulk-type piezoelectric member. Therefore, it is possible to mount the ultrasonic probe on the low-profile housing as described above, and it becomes possible to dispose the ultrasonic probe in the living body without damaging the living body.

An ultrasonic apparatus according to an application example of the invention includes the ultrasonic probe described above, and an image forming section adapted to form an image based on a signal output from the ultrasonic probe.

In this application example, the image is formed by the image forming section based on the signal from the ultrasonic probe obtained by the transmission process of the ultrasonic wave by the ultrasonic probe and the reception process of the reflected ultrasonic wave. Thus, it is possible for the practitioner to perform the operation to the object while checking the image formed by the image forming section.

In the ultrasonic apparatus according to the application example, it is preferable that the ultrasonic apparatus further includes an attitude detection section adapted to detect an attitude of the ultrasonic probe, and the image forming section one of rotates and flips the image in accordance with the attitude of the ultrasonic probe.

In the application example with this configuration, there is provided the attitude detection section adapted to detect the attitude of the ultrasonic probe, and the image is rotated or flipped in accordance with the attitude detected by the attitude detection section.

Thus, it is possible to correct the image formed by the image forming section to the orientation easy for the practitioner to check irrespective of the orientation at the time of arrangement of the ultrasonic probe, and it is possible to assist the efficient operation.

### BRIEF DESCRIPTION OF THE DRAWINGS

The invention will be described with reference to the accompanying drawings, wherein like numbers reference like elements.

FIG. 1 is a block diagram showing a schematic configuration of an ultrasonic apparatus according to an embodiment of the invention.

FIG. 2 is a perspective view showing a schematic configuration of an ultrasonic probe in the ultrasonic apparatus according to the embodiment.

FIG. 3A is a side view of the ultrasonic probe, and FIG. 3B is a cross-sectional view of a vicinity of a connection part of a hand part.

FIG. 4 is a plan view of an ultrasonic sensor according to the embodiment.

FIG. 5 is a cross-sectional view in a part of the ultrasonic sensor according to the embodiment.

FIG. 6 is a diagram showing a schematic configuration of a second hand part according to the present embodiment.

FIG. 7A is a diagram showing an ultrasonic scan plane when disposing the ultrasonic probe on an upper surface side with respect to a measurement object with the hand part located on a front side, and FIG. 7B is a diagram showing an image obtained with respect to the ultrasonic scan plane.

FIG. 8A is a diagram showing the ultrasonic scan plane when disposing the ultrasonic probe on a reverse surface side with respect to the measurement object with the hand part located on the front side, FIG. 8B is a diagram showing an image obtained with respect to the ultrasonic scan plane, and FIG. 8C is a diagram showing an internal tomographic image processed by an image forming section.

FIG. 9A is a diagram showing the ultrasonic scan plane when disposing the ultrasonic probe on a right side surface with respect to the measurement object with the hand part located on the front side, FIG. 9B is a diagram showing an image obtained with respect to the ultrasonic scan plane, and FIG. 9C is a diagram showing an internal tomographic image processed by the image forming section.

FIG. 10A is a diagram showing the ultrasonic scan plane when disposing the ultrasonic probe on a left side surface with respect to the measurement object with the hand part located on the front side, FIG. 10B is a diagram showing an image obtained with respect to the ultrasonic scan plane, and FIG. 10C is a diagram showing an internal tomographic image processed by the image forming section.

FIG. 11A is a diagram showing the ultrasonic scan plane when disposing the ultrasonic probe on the upper surface side with respect to the measurement object with the hand part located on a back side, FIG. 11B is a diagram showing an image obtained with respect to the ultrasonic scan plane, and FIG. 11C is a diagram showing an internal tomographic image processed by the image forming section.

FIG. 12A is a diagram showing the ultrasonic scan plane when disposing the ultrasonic probe on the reverse surface side with respect to the measurement object with the hand part located on the back side, FIG. 12B is a diagram showing an image obtained with respect to the ultrasonic scan plane, and FIG. 12C is a diagram showing an internal tomographic image processed by the image forming section.

FIG. 13A is a diagram showing the ultrasonic scan plane when disposing the ultrasonic probe on the right side surface with respect to the measurement object with the hand part located on the back side, FIG. 13B is a diagram showing an image obtained with respect to the ultrasonic scan plane, and FIG. 13C is a diagram showing an internal tomographic image processed by the image forming section.

FIG. 14A is a diagram showing the ultrasonic scan plane when disposing the ultrasonic probe on the left side surface with respect to the measurement object with the hand part located on the back side, FIG. 14B is a diagram showing an image obtained with respect to the ultrasonic scan plane, and FIG. 14C is a diagram showing an internal tomographic image processed by the image forming section.

FIG. 15A is a diagram showing the ultrasonic scan plane when disposing the ultrasonic probe on the back side with respect to the measurement object with the hand part located on the vertically upper side, and FIG. 15B is a diagram showing an image obtained with respect to the ultrasonic scan plane.

FIG. 16A is a diagram showing the ultrasonic scan plane when disposing the ultrasonic probe on the front side with respect to the measurement object with the hand part located on the vertically upper side, FIG. 16B is a diagram showing an image obtained with respect to the ultrasonic scan plane, and FIG. 16C is a diagram showing an internal tomographic image processed by the image forming section.

FIG. 17A is a diagram showing the ultrasonic scan plane when disposing the ultrasonic probe on the right side surface with respect to the measurement object with the hand part located on the vertically upper side, FIG. 17B is a diagram showing an image obtained with respect to the ultrasonic scan plane, and FIG. 17C is a diagram showing an internal tomographic image processed by the image forming section.

FIG. 18A is a diagram showing the ultrasonic scan plane when disposing the ultrasonic probe on the left side surface with respect to the measurement object with the hand part located on the vertically upper side, FIG. 18B is a diagram showing an image obtained with respect to the ultrasonic scan plane, and FIG. 18C is a diagram showing an internal tomographic image processed by the image forming section.

### DESCRIPTION OF AN EXEMPLARY EMBODIMENT

An embodiment of the invention will hereinafter be described with reference to the accompanying drawings.

### Schematic Configuration of Ultrasonic Apparatus 1

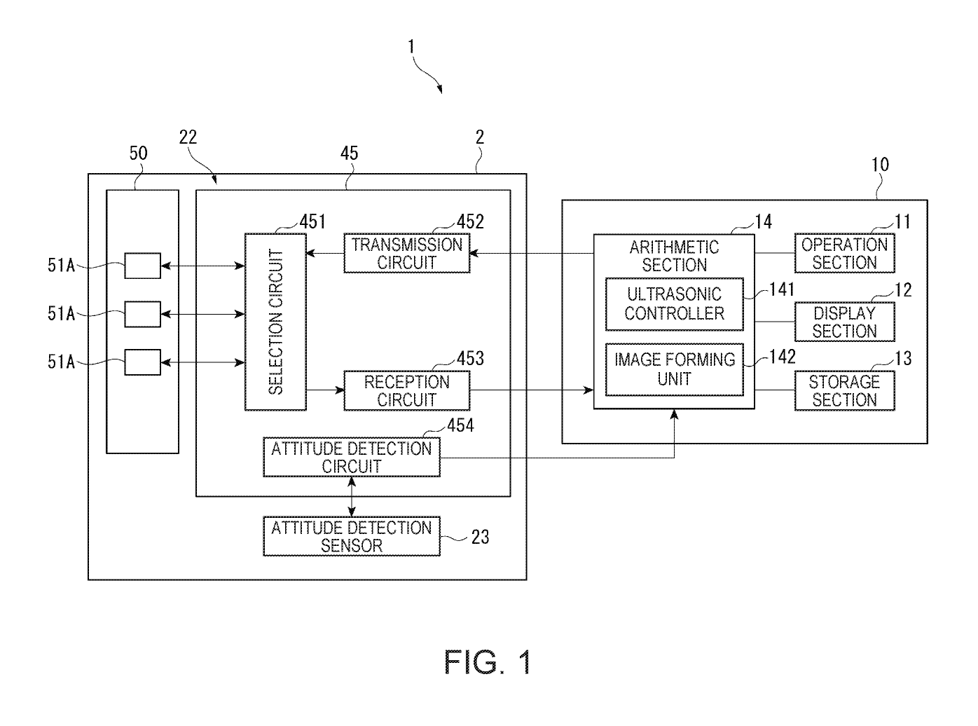

FIG. 1 is a block diagram showing a schematic configuration of an ultrasonic apparatus 1 according to the present embodiment.

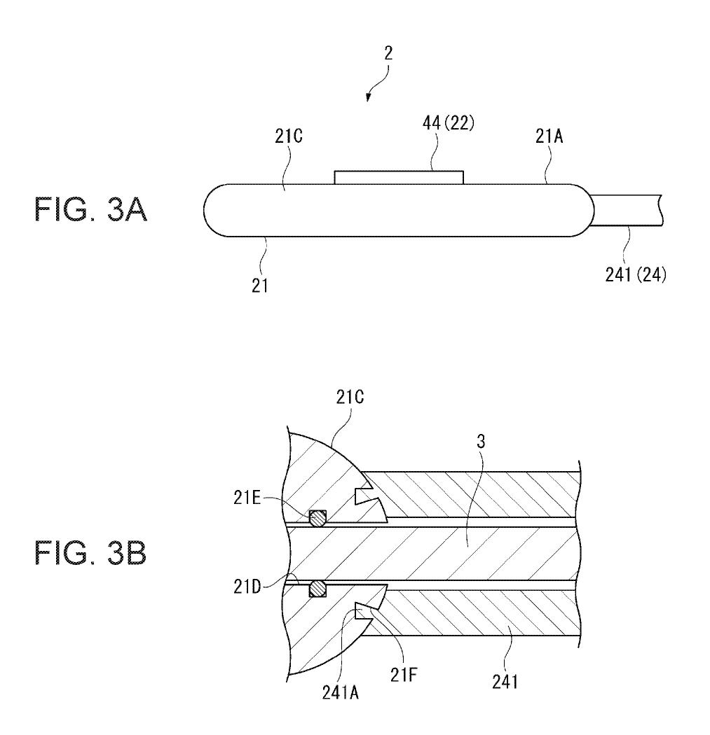

As shown in FIG. 1, the ultrasonic apparatus 1 according to the present embodiment is provided with an ultrasonic probe 2, and a control device 10 electrically connected to the ultrasonic probe 2 via a cable 3 (see FIGS. 3A and 3B).

The ultrasonic apparatus 1 transmits an ultrasonic wave from the ultrasonic probe 2 to the inside of a living body with the ultrasonic probe 2 having contact with a surface of a part (e.g., an organ) of a measurement object in the living body. Further, the ultrasonic apparatus 1 receives the ultrasonic wave reflected by the part of the living body using the ultrasonic probe 2, and then obtains, for example, an internal tomographic image in the living body based on the received signal. The ultrasonic apparatus 1 is mainly used by a practitioner for checking the internal tomographic image of the measurement object (an operation object and a part in the vicinity of the operation object) when the practitioner performs an operation taking the part in the living body as the operation object in a medical institution or the like.

### Configuration of Ultrasonic Probe 2

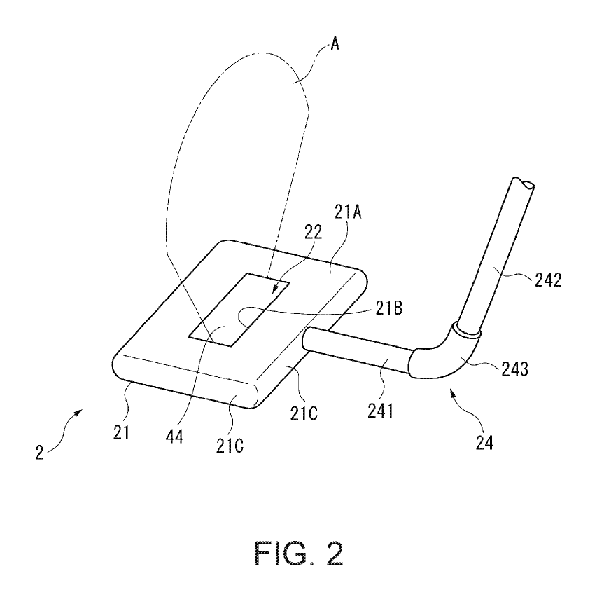

FIG. 2 is a perspective view showing a schematic configuration of the ultrasonic probe in the ultrasonic apparatus according to the embodiment. FIG. 3A is a side view of the ultrasonic probe, and FIG. 3B is a cross-sectional view of a vicinity of a connection part of a hand part 24.

As shown in FIG. 2, and FIGS. 3A and 3B, the ultrasonic probe 2 is configured including a housing 21, an ultrasonic sensor 22, an attitude detection sensor 23 (see FIG. 1), and the hand part 24.

As shown in FIG. 2, the housing 21 is configured so as to have a thin box-like shape, and is formed to have a thickness dimension equal to or smaller than 1 cm. A surface perpendicular to the thickness direction of the housing 21 constitutes a sensor surface 21A (a transmitting/receiving surface). The sensor surface 21A is provided with a sensor window 21B, and a part (an acoustic lens 44) of the ultrasonic sensor 22 is exposed from the sensor window 21B. Further, a side surface 21C adjacent to the sensor surface 21A of the housing 21 is configured to be a convexly curved surface, and suppresses damage to the living body when touching the living body.

Further, the hand part 24 is disposed in a part of the side surface 21C of the housing 21. The detailed explanation of the hand part 24 will be described later.

### Configuration of Ultrasonic Sensor 22

The ultrasonic sensor 22 is disposed on the sensor surface 21A side in the inside of the housing 21.

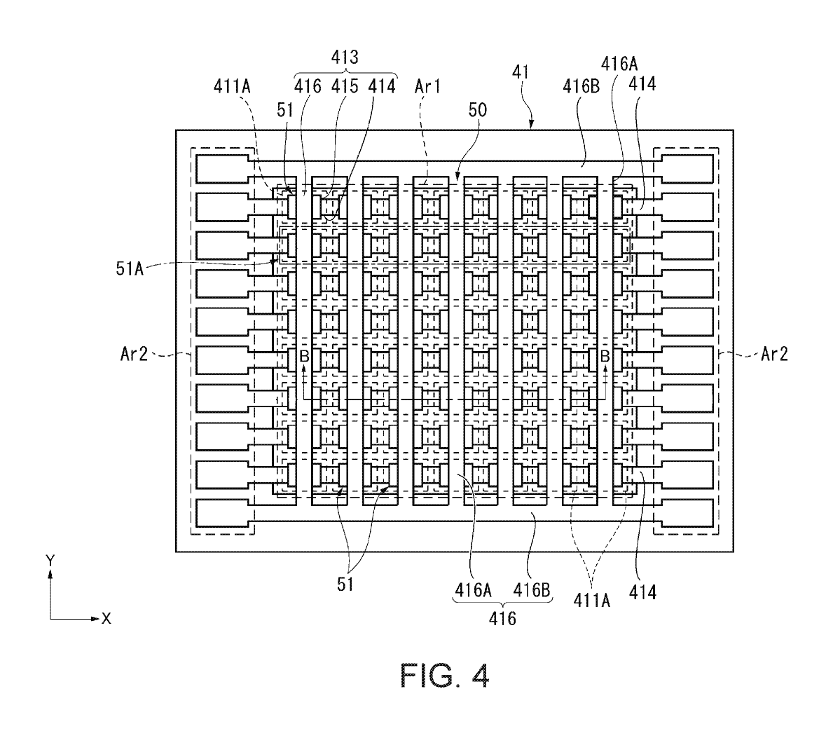

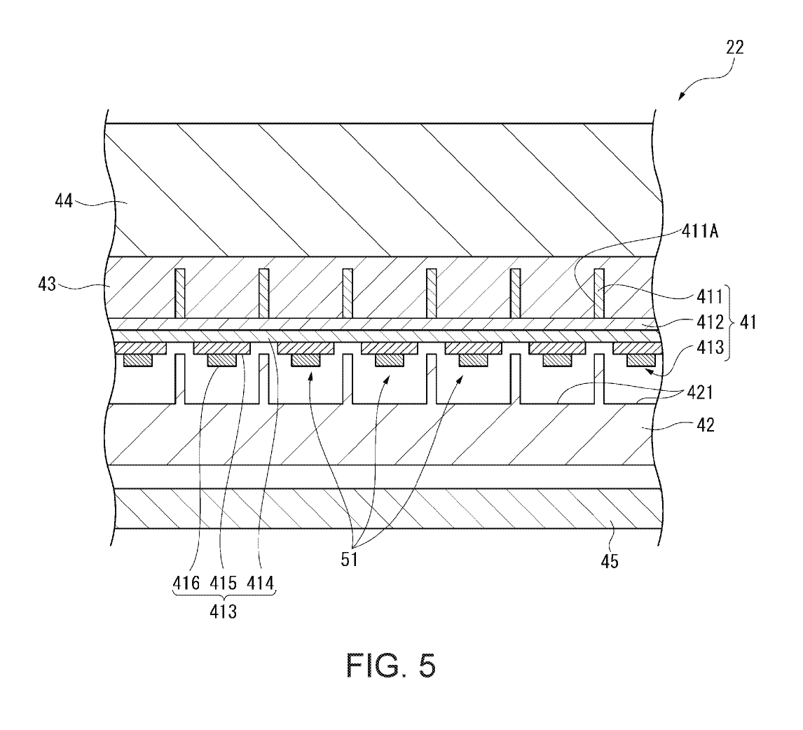

FIG. 4 is a plan view of the ultrasonic sensor 22, and FIG. 5 is a cross-sectional view (a cross-sectional view as a cutting surface along the line B-B shown in FIG. 4) of a part of the ultrasonic sensor 22.

As shown in FIG. 5, the ultrasonic sensor 22 is provided with an element substrate 41, a sealing plate 42, an acoustic matching layer 43, the acoustic lens 44, and a wiring board 45.

The element substrate 41 is provided with a substrate main body part 411, a support film 412 stacked on the substrate main body part 411, and piezoelectric elements 413 (vibrators) stacked on the support film 412.

The element substrate 41 is provided with an array region Ar1 disposed in a central part of the substrate in a planar view viewed from the thickness direction. The array region Ar1 is provided with a plurality of ultrasonic transducers 51 arranged in an array to constitute an ultrasonic transducer array 50. Further, outside the array region Ar1 of the element substrate 41, there are disposed terminal regions Ar2 to which electrode lines connected to the respective ultrasonic transducers 51 are extracted.

The substrate main body part 411 is, for example, a semiconductor substrate made of, for example, Si. Inside the array region Ar1 of the substrate main body part 411, there are disposed opening parts 411A corresponding respectively to the ultrasonic transducers 51. Further, on one surface of the substrate main body part 411, there is disposed the support film 412, and each of the opening parts 411A is blocked by the support films 412.

The support film 412 is formed of, for example, SiO2 or a laminated body of SiO2 and ZrO2, and blocks one ends of the opening parts 411A as described above.

The piezoelectric elements 413 are disposed on the support film 412 for blocking each of the opening parts 411A, and are each formed of a laminated body of a lower part electrode 414, a piezoelectric film 415, and an upper part electrode 416. Here, the support film 412 and a piezoelectric element 413 constitute the ultrasonic transducer 51 according to the invention.

In such an ultrasonic transducer 51, by applying a rectangular-wave voltage having a predetermined frequency between the lower part electrode 414 and the upper part electrode 416, it is possible to vibrate the support film 412 in an opening region of each of the opening parts 411A to transmit the ultrasonic wave. Further, when the support film 412 is vibrated due to a reflected ultrasonic wave, a potential difference is generated between an upper part and a lower part of the piezoelectric film 415, and it becomes possible to detect the ultrasonic wave thus received by detecting the potential difference generated between the lower part electrode 414 and the upper part electrode 416.

Further, in the present embodiment, as shown in FIG. 4, a plurality of such ultrasonic transducers 51 as described above is arranged in the array region Ar1 of the element substrate 41 along an X direction (a first direction) and a Y direction (a second direction) perpendicular to the X direction.

Here, the lower part electrodes 414 are each formed to have a straight-line shape along the X direction, and are each disposed straddling the plurality of ultrasonic transducers 51 arranged along the X-direction. Further, an end part of each of the lower part electrodes 414 extends to the terminal region Ar2, and is electrically connected to the wiring substrate 45 in the terminal region Ar2.

On the other hand, the upper part electrode 416 is provided with first upper part electrodes 416A each disposed straddling the plurality of ultrasonic transducers 51 arranged side by side along the Y direction, and second upper part electrodes 416B each connecting end parts of the first upper part electrodes 416A to each other. An end part of each of the second upper part electrodes 416B extends to the terminal region Ar2, and is electrically connected to the wiring substrate 45 in the terminal region Ar2.

In such an ultrasonic transducer array 50 as described above, there is formed a one-dimensional array structure in which the ultrasonic transducers 51 connected by the lower part electrode 414 to each other and arranged in the X direction constitute one ultrasonic transducer group 51A, and the plurality of ultrasonic transducer groups 51A is arranged along the Y direction.

The sealing plate 42 is disposed for reinforcing the strength of the element substrate 41, and is formed of a metal plate made of, for example, 42 alloy, a semiconductor substrate, or the like, and is bonded to the element substrate 41. The material and the thickness of the sealing plate 42 affect the frequency characteristics of the ultrasonic transducer 51, and are therefore preferably set based on the central frequency of the ultrasonic wave transmitted/received by the ultrasonic transducer 51.

Further, the sealing plate 42 is provided with concave grooves 421 disposed at positions overlapping the opening parts 411A of the element substrate 41 in a planar view, respectively. The concave grooves 421 are provided for suppressing the influence of a back wave of the ultrasonic wave generated by the vibration of the support film 412. In other words, each of the concave grooves 421 suppresses the problem (cross talk) that the back wave generated in one ultrasonic transducer 51 is input to another ultrasonic transducer 51 adjacent to the one ultrasonic transducer 51. Further, the groove depth of the concave grooves 421 is set to an odd multiple of a quarter (λ/4) of the wavelength λ of the ultrasonic wave. Thus, when the back wave reflected by the sealing plate 42 is input again to the ultrasonic transducer 51, the phase shift with the ultrasonic wave emitted from the ultrasonic transducer 51 toward the living body side (the opposite side to the sealing plate 42) is suppressed to suppress the attenuation of the ultrasonic wave.

As shown in FIG. 5, the acoustic matching layer 43 is disposed on a surface of the element substrate 41, the surface being located on the opposite side to the sealing plate 42. Specifically, the acoustic matching layer 43 is formed so as to fill the opening parts 411A of the element substrate 41, and to have a predetermined thickness dimension from the surface of the substrate main body part 411.

The acoustic lens 44 is disposed on the acoustic matching layer 43, and as shown in FIG. 2, and FIGS. 3A and 3B, exposed to the outside from the sensor window 21B of the housing 21.

The acoustic matching layer 43 and the acoustic lens 44 efficiently propagate the ultrasonic wave emitted from the ultrasonic transducer 51 to the living body as the measurement object, and further propagate the ultrasonic wave, which has been reflected in the living body, to the ultrasonic transducer 51 with efficiency. Therefore, the acoustic matching layer 43 and the acoustic lens 44 set to have an acoustic impedance intermediate between the acoustic impedance of the ultrasonic transducer 51 of the element substrate 41 and the acoustic impedance of the living body.

The wiring board 45 is a board to which the element substrate 41 and the sealing plate 42 are fixed, and has a terminal part (not shown) to be connected to the electrode lines (the lower part electrodes 414, the upper part electrode 416) extracted to the terminal region Ar2 of the element substrate 41. As the connection between the electrode lines and the terminal part, there can be cited connection using, for example, flexible printed circuits (FPC), or through electrodes disposed so as to penetrate the sealing plate 42.

Further, the wiring board 45 is provided with a driver circuit for driving each of the ultrasonic transducers of the ultrasonic transducer array 50, and so on. Specifically, as shown in FIG. 1, the wiring board 45 is provided with a selection circuit 451, a transmission circuit 452, a reception circuit 453, an attitude detection circuit 454, and so on.

It should be noted that in the present embodiment, the upper part electrode 416 is common to the ultrasonic transducers 51. Therefore, in the present embodiment, in the wiring board 45, the terminal part connected to the upper part electrode 416 is connected to, for example, a ground circuit, and is set to a predetermined common potential (e.g., zero potential).

The selection circuit 451 switches between transmission connection of connecting the ultrasonic sensor 22 and the transmission circuit 452 to each other, and reception connection of connecting the ultrasonic sensor 22 and the reception circuit 453 to each other based on the control of the control device 10.

The transmission circuit 452 outputs a transmission signal, which represents that the ultrasonic sensor 22 is made to transmit the ultrasonic wave, via the selection circuit 451 when switching to the transmission connection is performed due to the control of the control device 10.

The reception circuit 453 outputs a reception signal, which is input from the ultrasonic sensor 22, to the control device 10 via the selection circuit 451 when switching to the reception connection is performed due to the control of the control device 10. The reception circuit 453 is configured including, for example, a low-noise amplifier circuit, a voltage-controlled attenuator, a programmable-gain amplifier, a low-pass filter, and an A/D converter, and performs a variety of signal processing such as conversion of the reception signal to a digital signal, elimination of a noise component, and amplification to a desired signal level, and then outputs the reception signal thus processed to the control device 10.

The attitude detection circuit 454 is connected to the attitude detection sensor 23, and performs drive control of the attitude detection sensor 23 and attitude detection of the ultrasonic probe 2 (detection of the position and the orientation of the sensor surface 21A). In other words, the attitude detection circuit 454 constitutes an attitude detection section according to the invention together with the attitude detection sensor 23. Further, the attitude detection circuit 454 outputs the attitude (an attitude detection signal) of the ultrasonic probe 2 thus detected to the control device 10.

### Configuration of Attitude Detection Sensor 23

The attitude detection sensor 23 is disposed inside the housing 21, and constitutes an attitude detection section according to the invention together with the attitude detection circuit 454. The attitude detection sensor 23 is formed of, for example, a gyro sensor or an acceleration sensor, and detects the attitude (the direction to which the sensor surface 21A faces) of the ultrasonic probe 2.

Further, the attitude detection sensor 23 is connected to the attitude detection circuit 454 of the wiring board 45, and the attitude (the attitude detection signal) of the ultrasonic probe 2 thus detected is transmitted from the attitude detection circuit 454 to the control device 10.

### Configuration of Hand Part 24

As shown in FIG. 2, and FIGS. 3A and 3B, the hand part 24 is connected to the side surface 21C of the housing 21.

The hand part 24 is provided with a first hand part 241 connected to the side surface 21C of the housing 21, and a second hand part 242 connected to the first hand part 241 via a connection part 243.

The first hand part 241 is a shaft member having an axial direction parallel to the sensor surface 21A as the transmitting/receiving surface for the ultrasonic wave of the housing 21. The first hand part 241 is formed to have a cylindrical shape, and a hollow shape is formed inside due to the cylindrical inner peripheral surface. The cable 3 is inserted into the hollow shaft core part formed by the cylindrical inner peripheral surface.

More specifically, the side surface 21C of the housing 21 is provided with a through hole 21D (see FIG. 3B) for the cable 3, and the cable 3 for connecting the wiring board 45 of the ultrasonic sensor 22 and the control device 10 to each other is drawn through the through hole 21D. It should be noted that a gap between the through hole 21D and the cable 3 is provided with a waterproof member 21E such as a packing member or resin. Further, the first hand part 241 is connected to the side surface 21C of the housing 21 so as to be coaxial with the through hole 21D.

The first hand part 241 is detachably attached to the side surface 21C so that the hollow part having a cylindrical inner periphery becomes coaxial with the through hole 21D. The method of fixing the first hand part 241 to the side surface 21C is not particularly limited, as shown in, for example, FIG. 3B, there can be cited a configuration in which an engaging claw 241A disposed at one end of the first hand part 241 is made to engage a locking part 21F provided to the side surface 21C, and so on.

Further, by disposing the engaging claw 241A and the locking part 21F so as to have a ring-like shape coaxial with the first hand part 241 (the through hole 21D), it is also possible to obtain a configuration in which the first hand part 241 can rotate around the shaft core.

A second hand part 242 is formed to have a hollow shape inside (cylindrical inner periphery) similarly to the first hand part 241, and is connected to the first hand part 241 via the connection part 243. Further, the second hand part 242 can also be disposed so as to detachably be attached to the connection part 243.

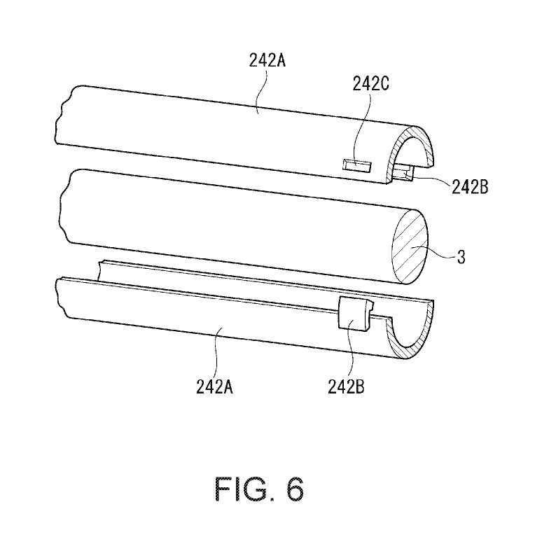

As a configuration of attaching and detaching the second hand part 242, the configuration shown in FIG. 6, for example, can be cited. FIG. 6 is a diagram showing a schematic configuration of the second hand part 242.

Specifically, the second hand part 242 is provided with a plurality of (e.g., two) partial hand parts 242A each elongated along the axial direction and having a U cross-sectional shape. These partial hand parts 242A are each provided with an engaging part 242B and an engaging groove 242C disposed on the outer peripheral surface, and are coupled to each other by engaging these engaging parts 242B and the engaging grooves 242C with each other, and can be decoupled by releasing the engagement. It should be noted that it is also possible to dispose a plurality of engaging parts 242B and engaging grooves 242C.

Further, the configuration of attaching and detaching the second hand part 242 is not limited to the above. In the case in which there is adopted a configuration in which the second hand part 242 is detachably attached to the connection part 243 it is also possible to adopt a configuration of, for example, sliding the second hand part 242 along the cable 3 inserted on the shaft core in a direction of getting away from the connection part 243 to detach the second hand part 242.

The connection part 243 is formed of, for example, flexible resin, and as shown in FIG. 2, connects the first hand part 241 and the second hand part 242 to each other so that the angle between the first hand part 241 and the second hand part 242 can be changed. The connection part 243 has a hollow shape through which the cable 3 is inserted similarly to the first hand part 241 and the second hand part 242.

It should be noted that in FIG. 2, although the example in which the connection part 243 is formed of the flexible tube is illustrated as the connection part 243, the connection part 243 is not limited to this configuration. For example, it is also possible to adopt a configuration in which by further providing a ratchet mechanism and so on, the angle of the second hand part 242 to the first hand part 241 can be kept at a predetermined angle. On this occasion, in the case in which there is adopted a configuration in which the first hand part 241 can rotate around the shaft core as described above, the axial direction of the second hand part 242 can arbitrarily be changed to an arbitrary direction.

Further, the connection part 243 can be provided with a configuration in which the connection part 243 is detachably attached to the first hand part 241 and the second hand part 242. On this occasion, it becomes possible to replace the connection part 243 with one different in elastic coefficient in accordance with, for example, the measurement object, and it becomes possible to more efficiently install the ultrasonic probe 2.

### Configuration of Control Device 10

As shown in FIG. 1, the control device 10 is configured including, for example, an operation section 11, a display section 12, a storage section 13, and an arithmetic section 14. As the control device 10, there can be used a terminal device such as a tablet terminal, a smartphone, or a personal computer, and the control device 10 can also be a dedicated terminal device for operating the ultrasonic probe 2.

The operation section 11 is a user interface (UI) for the user to operate the ultrasonic apparatus 1, and can be formed of, for example, a touch panel or operation buttons disposed on the display section 12, a keyboard, or a mouse.

The display device 12 is formed of, for example, a liquid crystal display, and displays an image.

The storage section 13 stores a variety of programs and a variety of data for controlling the ultrasonic apparatus 1.

The arithmetic section 14 is formed of an arithmetic circuit such as a central processing unit (CPU), and a storage circuit such as a memory. Further, the arithmetic section 14 reads and executes the variety of programs stored in the storage section 13 to thereby function as an ultrasonic controller 141 and an image forming unit 142.

The ultrasonic controller 141 controls the ultrasonic sensor 22 to perform control of transmission/reception of the ultrasonic wave by the ultrasonic sensor 22. Specifically, the ultrasonic controller 141 outputs a drive signal to the lower part electrode 414 of each of the ultrasonic transducer groups 51A to drive the ultrasonic transducer groups 51A. On this occasion, by delaying the drive signal input to each of the ultrasonic transducer groups 51A, it becomes possible to control the transmission direction of the ultrasonic wave. Thus, as shown in FIG. 2, it becomes possible to transmit the ultrasonic wave to the inside of the ultrasonic scan plane A, which has a fan-like shape, includes a direction (the Y direction) in which the ultrasonic transducer groups 51A arranged in the ultrasonic sensor 22, and is perpendicular to the sensor surface 21A. Further, by receiving the ultrasonic wave, which has been reflected by the measurement object located on the ultrasonic scan plane A, in the ultrasonic sensor 22, it is possible to detect the position (a boundary part where a difference occurs in the acoustic impedance) where the ultrasonic wave has been reflected in the ultrasonic scan plane A.

The image forming unit 142 is an image formation section according to the invention, and forms an image (internal tomographic image in the living body) based on the reception signal of the ultrasonic wave having been processed by the reception circuit 453, and then makes the display section 12 display the image.

On this occasion, the image forming unit 142 reverses or rotates the image thus generated based on the attitude (the attitude detection signal based on the orientation of the sensor surface 21A and so on) of the ultrasonic probe 2 detected by the attitude detection sensor 23 and the attitude detection circuit 454.

### Ultrasonic Measurement Method Using Ultrasonic Apparatus 1

The measurement method of the measurement object using the ultrasonic apparatus 1 described above will hereinafter be described.

As described above, the ultrasonic apparatus 1 according to the embodiment is used for observing the internal tomographic image of the measurement object (an operation object and a part in the periphery of the operation object) when, for example, performing the operation to the operation object inside the living body.

Specifically, in the case in which the practitioner observes the internal tomographic image with respect to the measurement object inside the living body, the practitioner (or an operator of the ultrasonic apparatus 1) picks up the hand part 24 and then inserts the ultrasonic probe 2 to the side or the reverse of the measurement object. Then, the practitioner or the operator drives the ultrasonic apparatus 1. Thus, the ultrasonic controller 141 performs the transmission/reception process of the ultrasonic wave using the ultrasonic sensor 22. Further, the image forming unit 142 forms the internal tomographic image based on the ultrasonic wave thus received, and then makes the display section 12 display the internal tomographic image.

On this occasion, the image forming unit 142 processes the image thus formed based on the attitude of the ultrasonic probe 2 detected by the attitude detection sensor 23.

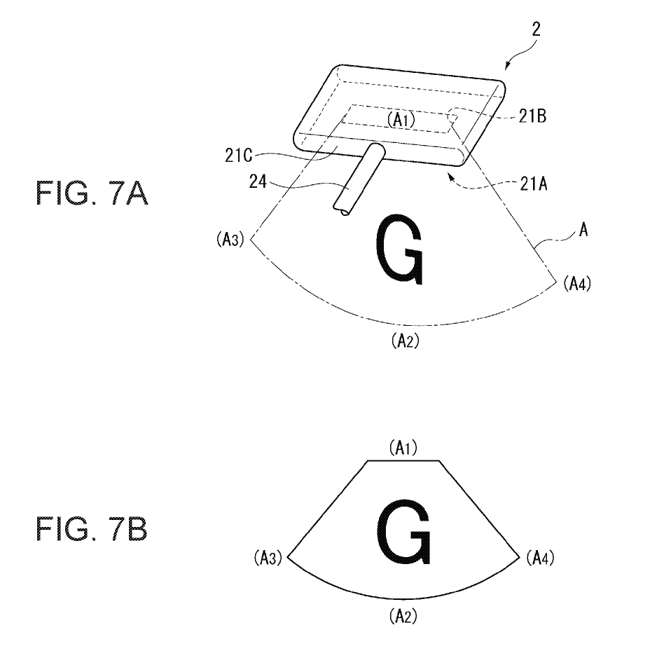

FIG. 7A is a diagram showing the ultrasonic scan plane A when pressing the ultrasonic probe 2 against the measurement object from the upper surface side, and FIG. 7B is a diagram showing an image obtained from the ultrasonic probe 2.

In the present embodiment, the image forming unit 142 determines the attitude, which is detected by the attitude detection sensor 23 when pressing the ultrasonic probe 2 against the upper surface of the measurement object with the hand part 24 located on the practitioner side (front side) as shown in FIG. 7A, as an attitude in a positive direction. In this case, the image forming unit 142 makes the display section 12 display the image (the internal tomographic image) of the ultrasonic scan plane A based on the received ultrasonic wave obtained by the ultrasonic probe 2 without reversing or rotating the image.

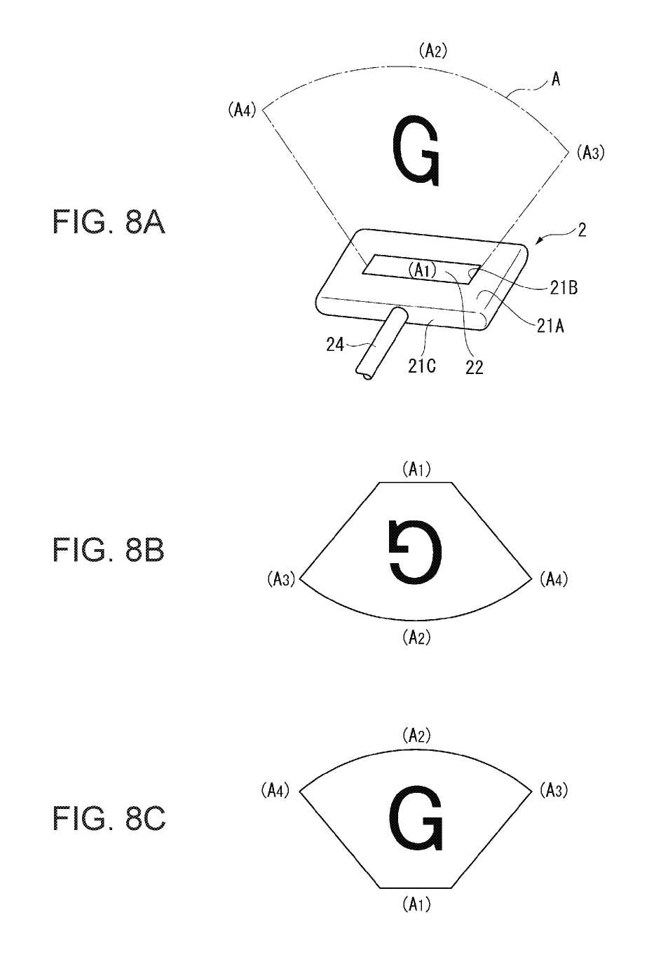

FIGS. 8A through 8C, FIGS. 9A through 9C, and FIGS. 10A through 10C are diagrams showing the attitude of the ultrasonic probe, the image obtained, and the image processed by the image forming unit 142, respectively, when observing the image of the tomographic plane (the ultrasonic scan plane A) the same as in FIG. 7A with the ultrasonic probe 2 disposed on the side and the reverse with the hand part 24 located on the practitioner side (the front side).

As shown in FIG. 8A, if the ultrasonic probe 2 is disposed on the reverse of the measurement object with the hand part 24 located on the front side, the attitude detection sensor 23 and the attitude detection circuit 454 detect the attitude of the ultrasonic probe 2, and then output the result to the control device 10. On the other hand, from the ultrasonic probe 2, there is input the ultrasonic received signal corresponding to the image shown in FIG. 8B. In this case, the image forming unit 142 performs a 180-degree rotation process (or a vertically/horizontally flipping process) on the image (FIG. 8B) generated based on the ultrasonic received signal. Thus, as shown in FIG. 8C, it becomes possible to display the same internal tomographic image as in the case of pressing the ultrasonic probe 2 against the measurement object from the upper surface as shown in FIGS. 7A and 7B.

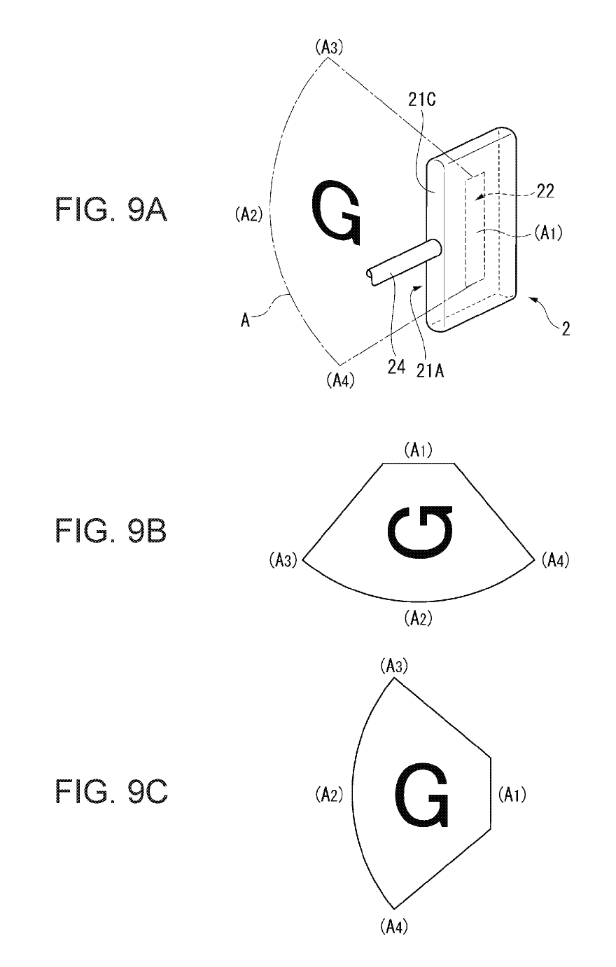

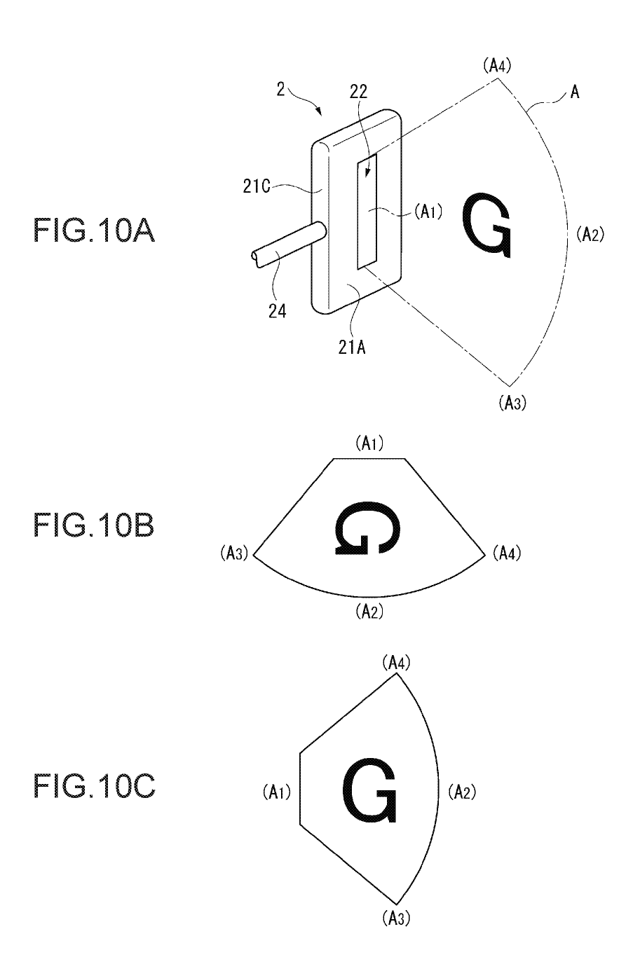

Roughly the same is also applies to such a case as shown in FIG. 9A through 9C and FIGS. 10A through 10C, in the case of, for example, FIGS. 9A through 9C, if the ultrasonic probe 2 is disposed on the right side of the measurement object with the hand part 24 located on the front side, the attitude detection sensor 23 and the attitude detection circuit 454 detect the attitude of the ultrasonic probe 2, and then output the result to the control device 10. In this case, the image forming unit 142 performs a rotation process clockwise as much as 90 degrees on the image (FIG. 9B) generated based on the ultrasonic received signal.

Further, in the case of FIGS. 10A through 10C, the attitude detection sensor 23 and the attitude detection circuit 454 detect the fact that the ultrasonic probe 2 is disposed on the left side of the measurement object with the hand part 24 located on the front side, and then output the result to the control device 10. In this case, the image forming unit 142 performs a rotation process counterclockwise as much as 90 degrees on the image (FIG. 10B) generated based on the ultrasonic received signal.

Thus, as shown in FIG. 9C and FIG. 10C, it becomes possible to display the same internal tomographic image (FIG. 7B) as in the case of pressing the ultrasonic probe 2 against the measurement object from the upper surface.

Further, although the above is the example in which the hand part 24 is disposed on the practitioner side (the front side), if the hand part 24 is disposed on the front side, the operation of the practitioner is hindered in some cases.

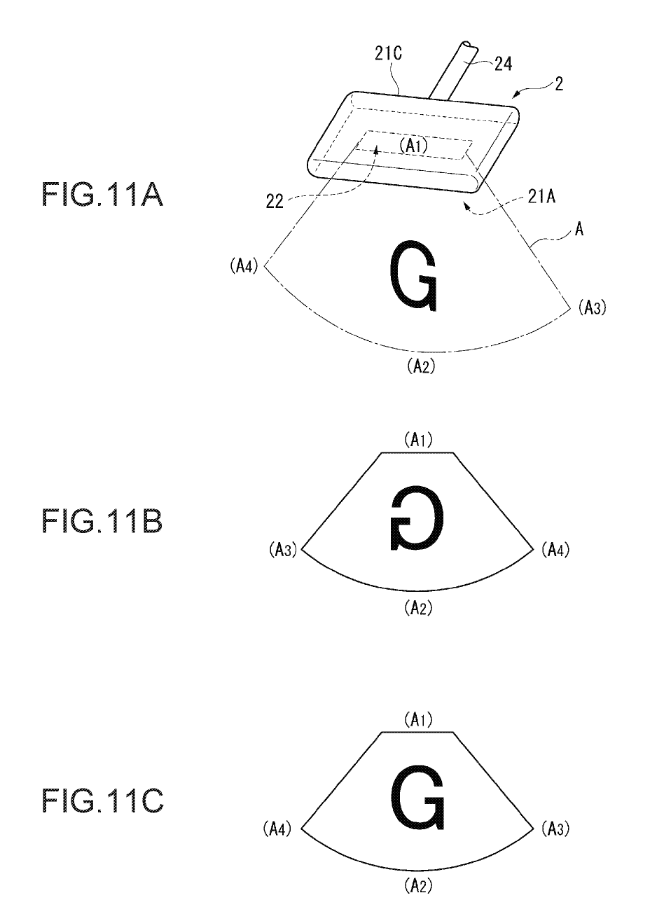

In such a case, it is sufficient to dispose the hand part 24 on the back side opposite to the practitioner side as shown in FIGS. 11A through 11C, FIGS. 12A through 12C, FIGS. 13A through 13C, and FIGS. 14A through 14C.

Here, in the example shown in FIGS. 11A through 11C, the attitude detection sensor 23 and the attitude detection circuit 454 detect the fact that the ultrasonic probe 2 is disposed on the upper surface of the measurement object with the hand part 24 located on the back side, and then output the result to the control device 10. In this case, the image forming unit 142 performs a horizontally flipping process on the image (FIG. 11B) generated based on the ultrasonic received signal.

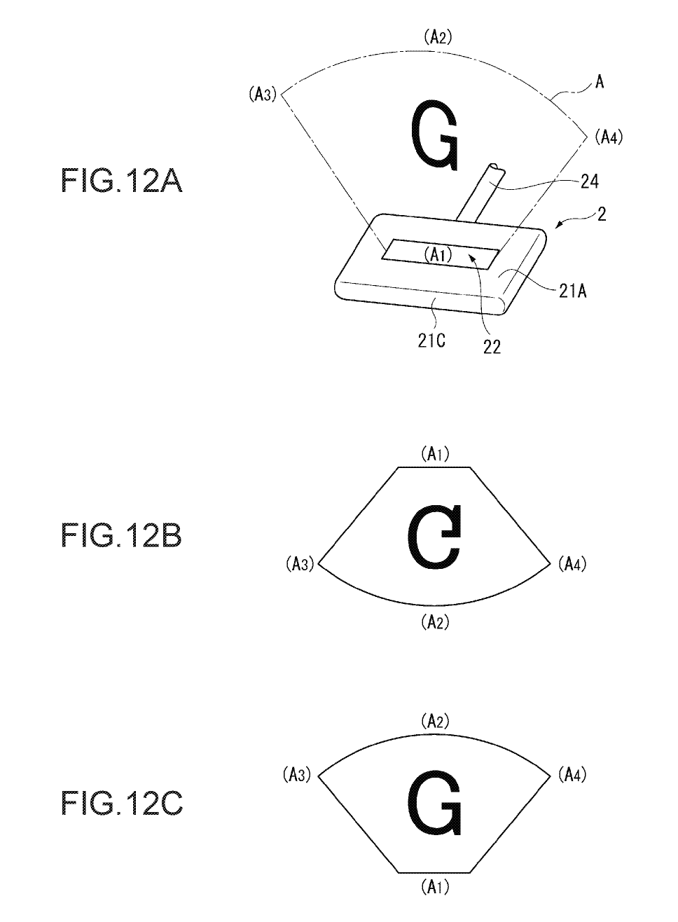

In the example shown in FIGS. 12A through 12C, the attitude detection sensor 23 and the attitude detection circuit 454 detect the fact that the ultrasonic probe 2 is disposed on the reverse surface of the measurement object with the hand part 24 located on the back side, and then output the result to the control device 10. In this case, the image forming unit 142 performs a 180-degree rotation process and the horizontally flipping process (or a vertically flipping process) on the image (FIG. 12B) generated based on the ultrasonic received signal.

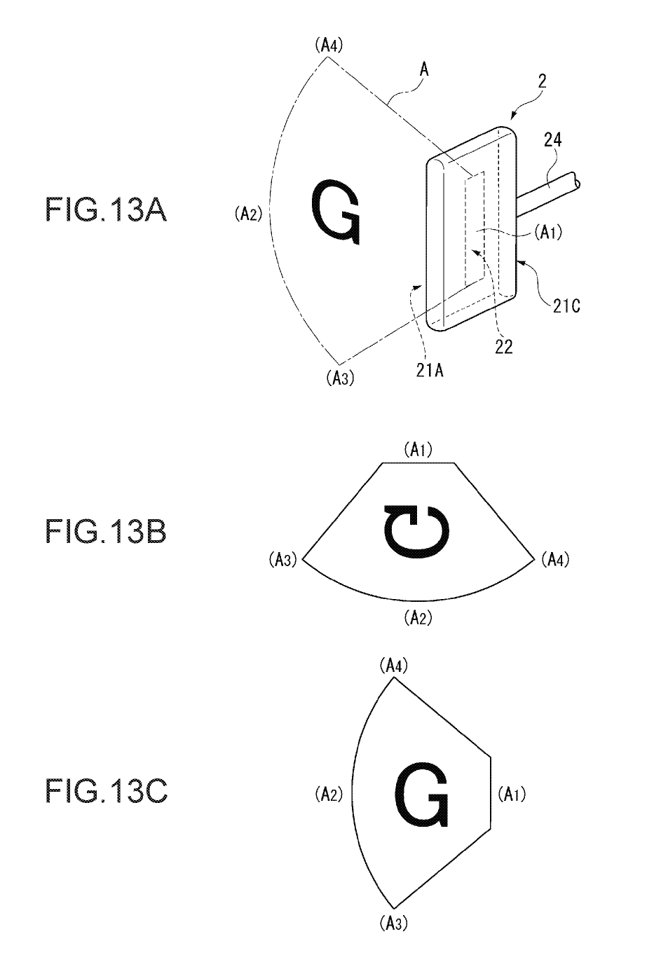

In the example shown in FIGS. 13A through 13C, the attitude detection sensor 23 and the attitude detection circuit 454 detect the fact that the ultrasonic probe 2 is disposed on the right side of the measurement object with the hand part 24 located on the back side, and then output the result to the control device 10. In this case, the image forming unit 142 performs a 90-degree counterclockwise rotation process and the horizontally flipping process (or a 90-degree clockwise rotation process and the vertically flipping process) on the image (FIG. 13B) generated based on the ultrasonic received signal.

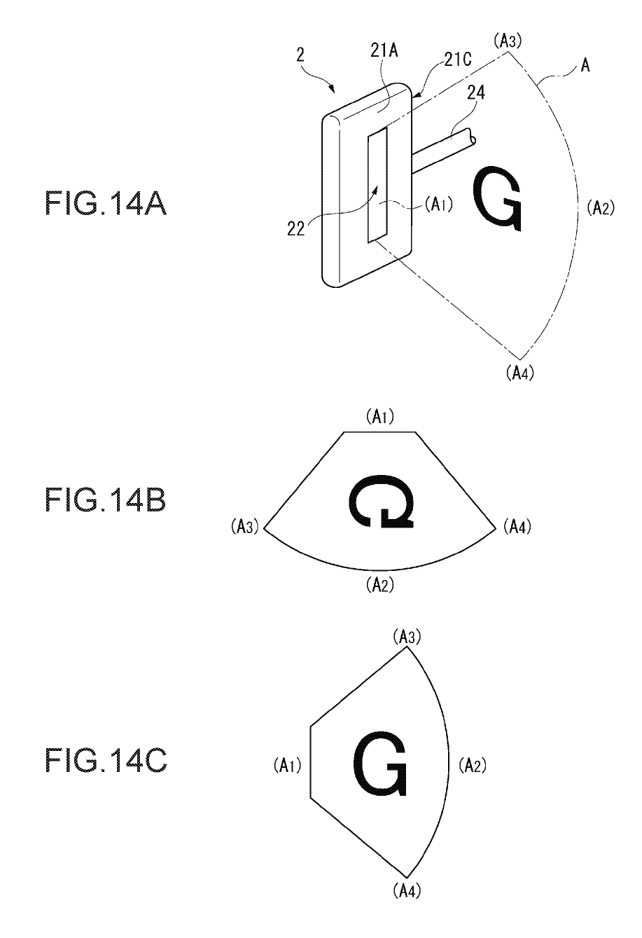

In the case of FIGS. 14A through 14C, the attitude detection sensor 23 and the attitude detection circuit 454 detect the fact that the ultrasonic probe 2 is disposed on the left surface of the measurement object with the hand part 24 located on the back side, and then output the result to the control device 10. In this case, the image forming unit 142 performs a 90-degree clockwise rotation process and the horizontally flipping process (or the 90-degree counterclockwise rotation process and the vertically flipping process) on the image (FIG. 14B) generated based on the ultrasonic received signal.

According to the above, as shown in FIG. 11C, FIG. 12C, FIG. 13C, and FIG. 14C, it becomes possible to display substantially the same image as the internal tomographic image shown in FIG. 7B.

In other words, it becomes possible to dispose the ultrasonic probe 2 so that the hand part 24 is located at a position where the hand part 24 does not interfere with the operation of the practitioner, and at the same time, it becomes possible to display the internal tomographic image of the measurement object viewed from the practitioner wherever the hand part 24 is located.

Incidentally, in the examples shown in FIGS. 7A, 7B, FIGS. 8A through 8C, FIGS. 9A through 9C, FIGS. 10A through 10C, FIGS. 11A through 11C, FIGS. 12A through 12C, FIGS. 13A through 13C, and FIGS. 14A through 14C, the ultrasonic probe 2 is disposed so that the hand part 24 is located on the front side or the back side of the practitioner to thereby set the ultrasonic scan plane A on the measurement cross section, for example, disposed along a roughly vertical direction and opposed to the practitioner. However, by arbitrarily setting the position of the hand part 24, the internal tomographic image in an arbitrary ultrasonic scan plane A can be obtained. For example, by locating the hand part 24 on the left side (or the right side) viewed from the practitioner, the ultrasonic scan plane A can be set in the direction of a plane disposed along a roughly vertical direction and perpendicular to the measurement cross section shown in FIG. 7A, FIG. 8A, FIG. 9A, FIG. 10A, FIG. 11A, FIG. 12A, FIG. 13A, and FIG. 14A.

Further, in the case in which the hand part 24 is located on the roughly vertically upper side of the housing 21 (in the case of, for example, holding the hand part 24 to insert the ultrasonic probe 2 to the side of the measurement object from the vertically upper side), the internal tomographic image in a roughly horizontal direction of the measurement object can be obtained.

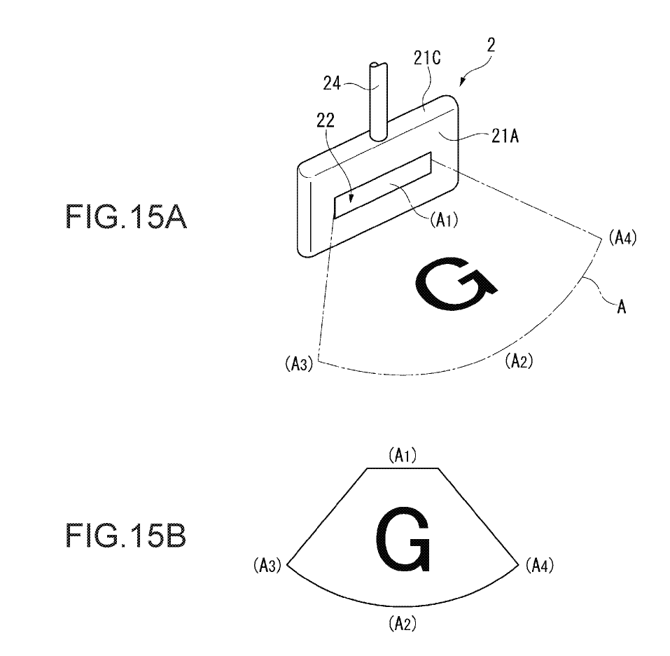

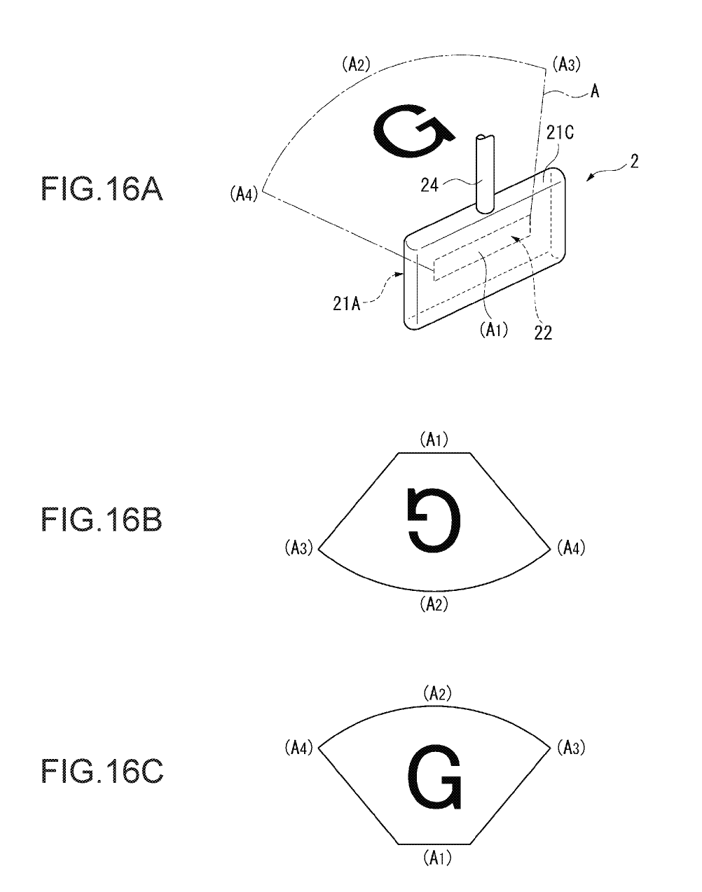

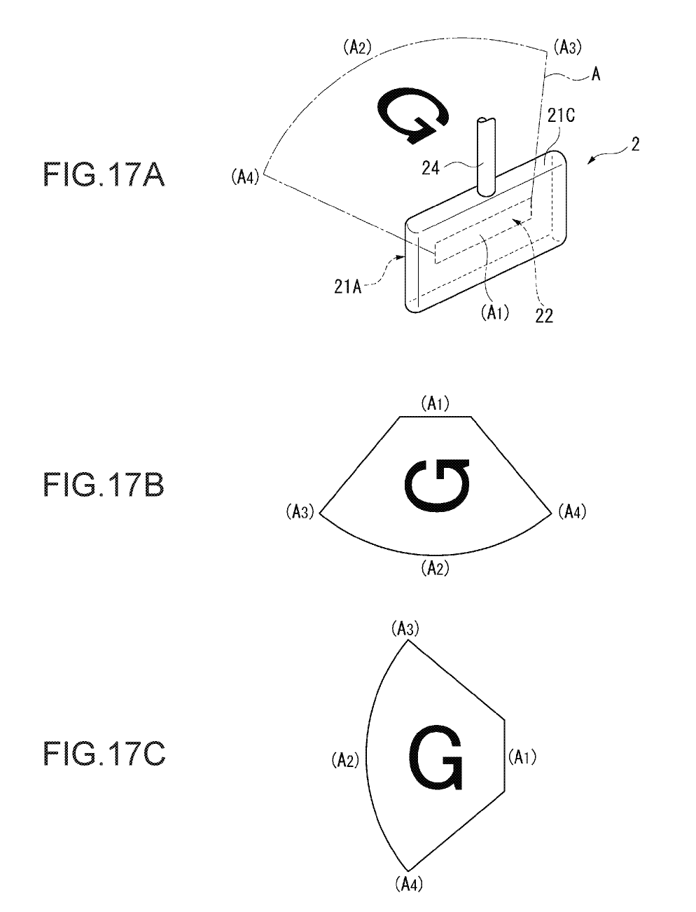

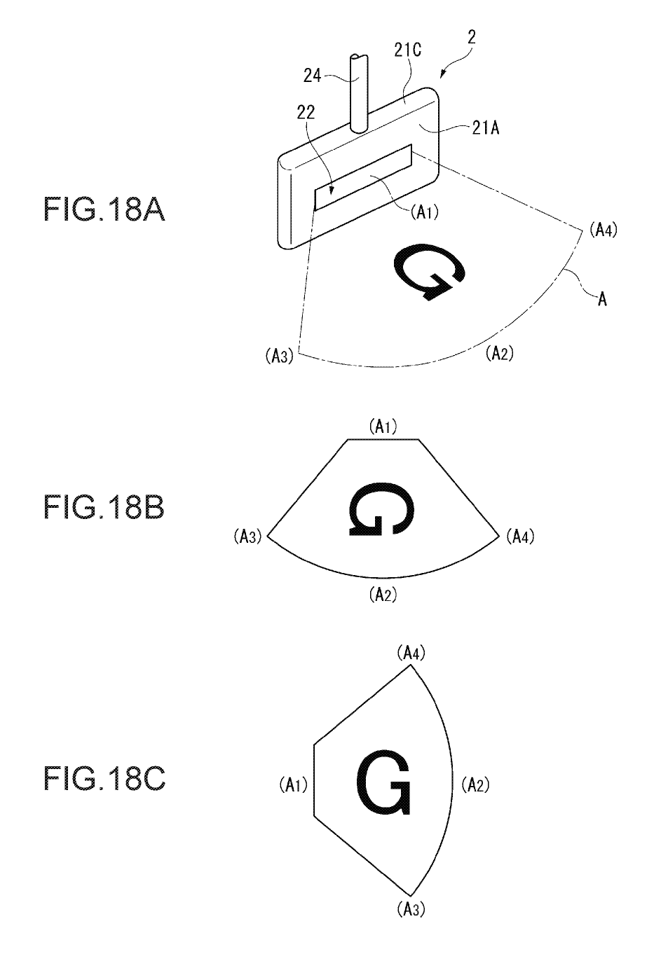

FIG. 15A, FIG. 16A, FIG. 17A, and FIG. 18A are arrangement examples of the ultrasonic probe 2 in the case of obtaining the internal tomographic image in the horizontal direction.

FIG. 15A shows the ultrasonic scan plane A when disposing the ultrasonic probe 2 to the opposite side (the back side) across the measurement object from the practitioner, and in this case, as shown in FIG. 15B, an image easy for the practitioner to check can be obtained from the received signal from the ultrasonic probe 2. Specifically, in this case, the image forming unit 142 makes the display section 12 display the image (the internal tomographic image) of the ultrasonic scan plane A based on the received ultrasonic wave obtained by the ultrasonic probe 2 without reversing or rotating the image.

In contrast, in the example shown in FIGS. 16A through 16C, the attitude detection sensor 23 and the attitude detection circuit 454 detect the fact that the ultrasonic probe 2 is disposed on the back side of the measurement object with the hand part 24 located on the vertically upper side, and then output the result to the control device 10. In this case, the image forming unit 142 performs the 180-degree rotation process (or the vertically/horizontally flipping process) on the image (FIG. 16B) generated based on the ultrasonic received signal.

In the example shown in FIGS. 17A through 17C, the attitude detection sensor 23 and the attitude detection circuit 454 detect the fact that the ultrasonic probe 2 is disposed on the right side surface of the measurement object with the hand part 24 located on the vertically upper side, and then output the result to the control device 10. In this case, the image forming unit 142 performs the 90-degree clockwise rotation process on the image (FIG. 17B) generated based on the ultrasonic received signal.

In the example shown in FIGS. 18A through 18C, the attitude detection sensor 23 and the attitude detection circuit 454 detect the fact that the ultrasonic probe 2 is disposed on the left side surface of the measurement object with the hand part 24 located on the vertically upper side, and then output the result to the control device 10. In this case, the image forming unit 142 performs the 90-degree counterclockwise rotation process on the image (FIG. 18B) generated based on the ultrasonic received signal.

According to the above, as shown in FIG. 16C, FIG. 17C, and FIG. 18C, it becomes possible to display substantially the same image as the internal tomographic image shown in FIG. 15B.

In other words, it becomes possible to dispose the ultrasonic probe 2 so that the hand part 24 is located at a position where the hand part 24 does not interfere with the operation of the practitioner, and at the same time, it becomes possible to display the internal tomographic image of the measurement object viewed from the practitioner wherever the hand part 24 is located.

Further, in the ultrasonic apparatus 1 according to the present embodiment, the practitioner or the operator is not required to take off the ultrasonic probe 2 after the arrangement of the ultrasonic probe 2 described above and the display of the internal tomographic image of the measurement object. That is, in the present embodiment, since the housing 21 of the ultrasonic probe 2 is disposed on the reverse or the side of the measurement object, the housing 21 does not hinder the operation. Further, the hand part 24 can also be moved toward the direction so as not hinder the operation. Therefore, it is possible to keep disposing the ultrasonic probe 2 without moving during the period in which the practitioner is performing the operation on the operation object, and thus it is possible to check the internal tomographic image of the measurement object using the ultrasonic apparatus 1 even during the operation.

Further, during the operation, it becomes possible to, for example, operate the hand part 24 to fine tune the position of the ultrasonic probe 2, and thus, it becomes possible to perform the operation high in efficiency.

### Functions and Advantages of Present Embodiment

In the ultrasonic apparatus 1 according to the present embodiment, the ultrasonic probe 2 is provided with the housing 21 having a thin plate shape incorporating the ultrasonic sensor 22, and the hand part 24 connected to the side surface 21C crossing the sensor surface 21A of the housing 21.

Therefore, in the state in which the operator picks up the hand part 24 and then inserts the ultrasonic probe 2 in the side or the reverse of the measurement object, the ultrasonic measurement (checking of the internal tomographic image) on the measurement object can be performed. Thus, in the living body operation, when the practitioner checks the internal tomographic image of the measurement object during the operation, an operation of suspending the operation to make the ultrasonic probe have contact with the measurement object, and so on become unnecessary, and the internal tomographic image of the measurement object can easily be checked. In particular in an operation to excise a liver tumor or the like, it is necessary to pay the closest attention not to cut an arterial vessel, and in the past, the check by the ultrasonic probe and the operation are performed alternately, or the like, which degrades the efficiency of the operation. In contrast, in the present embodiment, by disposing the ultrasonic probe 2 in the reverse or the side of the liver, the internal tomographic image of the liver can be checked with the ultrasonic wave anytime during the operation, and therefore, the efficiency of the operation can dramatically be improved.

In the ultrasonic probe 2 according to the present embodiment, the axial direction of the hand part 24 extends in a direction parallel to the plane direction of the sensor plane 21A from the side surface 21C of the housing 21. Therefore, when inserting the ultrasonic probe 2 into the reverse or the side of the measurement object, the problem that the hand part 24 exerts pressure on the part such as an organ as the measurement object and the periphery of the part, and so on do not arise, and the ultrasonic probe 2 can safely be disposed at a desired position.

The hand part 24 of the present embodiment is provided with the first hand part 241 and the second hand part 242, and the first hand part 241 is detachably attached to the housing 21.

Therefore, in the case in which the ultrasonic probe 2 is inserted to the side or the reverse of the measurement object, and then the hand part 24 hinders the operation, the first hand part 241 can be detached from the housing 21, and thus, an improvement in efficiency of the operation can be achieved.

Since the first hand part 241 is detachably attached to the housing 21, the first hand part 241 can arbitrarily be replaced with one different in axial length in accordance with the operation object. Further, cleansing of the gap between the first hand part 241 and the housing 21 can appropriately be performed.

Further, in the present embodiment, since the second hand part 242 is arranged to be changed in the angle with respect to the first hand part 241, in the case of, for example, disposing the ultrasonic probe 2 in the reverse of the measurement object, the efficiency of the insertion operation of the ultrasonic probe 2 is improved. Further, after grounding the ultrasonic probe 2, by changing the angle of the second hand part 242 to the first hand part 241, it is possible to move the second hand part 242 to the position where the second hand part 242 does not hinder the operation, and does not exert pressure on the part such as an organ.

Further, since the second hand part 242 is detachably attached to the connection part 243 for connecting the first hand part 241 and the second hand part 242 to each other, in the case in which the second hand part is an obstacle, the second hand part 242 can be detached.

Further, since the second hand part 242 is constituted by engaging the engaging parts 242B and the engaging grooves 242C of the pair of partial hand parts 242A with each other, by releasing the engagement state, the second hand part 242 can easily be detached. Further, when moving the ultrasonic probe 2, by engaging the engaging parts 242B and the engaging grooves 242C with each other, attachment can easily be achieved.

Further, the first hand part 241 can also be disposed so as to be rotatable with respect to the housing 21. In this case, even in the case in which a ratchet mechanism or the like is provided to the connection part 243 of the first hand part 241 and the second hand part 242, and the angle change is limited to a predetermined plane, by rotating the first hand part 241, the axial direction of the second hand part 242 can be moved to a desired direction.

In the present embodiment, the first hand part 241, the second hand part 242, and the connection part 243 are each formed to have the cylindrical internal surface, and are each provided with a hollow shaft core part, the cable 3 is inserted along the shaft core. Therefore, it is not necessary to separately extract the cable 3 from the housing 21. In other words, in the case of adopting the configuration of separately extracting the cable 3 from the housing 21, there is a possibility that the cable 3 hinders the operation. However, in the present embodiment, since the cable 3 passes through the center axis of the hand part 24 and is then extracted to the outside, there is no chance for the cable 3 to hinder the operation.

In the ultrasonic probe 2 according to the embodiment, the side surface 21C of the housing 21 has the convexly curved surface. Therefore, when inserting the ultrasonic probe 2 inside the living body, or after the insertion, the risk that the part in the living body is injured by an angular part of the housing 21 can be avoided, and thus, the ultrasonic probe 2 can safely be installed.

Further, since the thickness dimension of the housing 21 is no larger than 1 cm, even if the ultrasonic probe 2 is inserted in the living body, there is no chance for the ultrasonic probe 2 to exert pressure on the organ in the living body. Therefore, the ultrasonic probe 2 can safely be inserted in the living body.

The ultrasonic probe 2 of the present embodiment is provided with the ultrasonic transducers 51 each formed of the support film 412 and the piezoelectric element 413 having a film-like shape stacked on the support film 412 as the ultrasonic sensor 22 incorporated in the housing 21, and the plurality of ultrasonic transducers is arranged in an array to thereby constitute the ultrasonic transducer array 50.

As described above, the ultrasonic transducers 51 can constitute the ultrasonic sensor 22 having a thin plate-like shape using the support film 412 having the film-like shape and the piezoelectric elements 413 each having the film-like shape. In the ultrasonic probe using the bulk-type piezoelectric member having been used in the past, a back plate having a thickness dimension of 10 through 20 cm, for example, is necessary on the reverse side of the piezoelectric member, the size is too large to be embedded in the living body, and there is a risk of exerting pressure on other organs and so on. Therefore, it has been difficult to insert the ultrasonic probe to the reverse or the side of the measurement object as in the present embodiment. In contrast, in the present embodiment, as described above, since the ultrasonic sensor 22 having the ultrasonic transducers 51 each having the film-like shape and arranged in an array is used, the ultrasonic probe 2 can easily and safely be inserted in an necessary place in the living body as described above.

The ultrasonic apparatus 1 according to the present embodiment is provided with the image forming unit 142, and it is possible to form the internal tomographic image of the measurement object based on the transmission/reception process of the ultrasonic wave by the ultrasonic probe 2, and then make the display section 12 display the internal tomographic image. Therefore, in the operation to the living body and so on, it is possible for the practitioner to check the internal tomographic image of the operation object and the peripheral region anytime during the operation, and the efficiency of the operation can be improved.

Further, the ultrasonic probe 2 of the present embodiment is provided with the attitude detection sensor 23 formed of an acceleration sensor, a gyro sensor, or the like and the attitude detection circuit 454, and detects the attitude in the ultrasonic probe 2. In other words, what direction with respect to the housing 21 the hand part 24 extends, what direction the sensor surface 21A faces to, and so on are detected. Then, the image forming unit 142 processes the image generated based on the ultrasonic received signal using the rotation process, the horizontally flipping process, the vertically flipping process and so on so that the image becomes eye-friendly for the practitioner based on the attitude detection signal, and then makes the display section 12 display the image thus processed.

Therefore, it is possible to make the display section 12 display the internal tomographic image of the measurement object viewed from the practitioner irrespective of the position of the hand part 24 and the direction to which the sensor surface 21A faces. Therefore, it is possible for the practitioner to easily and appropriately check the internal structure of the measurement object, and the efficiency of the operation can further be improved.

### MODIFIED EXAMPLES

It should be noted that the invention is not limited to each of the embodiments described above, but includes modifications and improvements within a range where the advantages of the invention can be achieved, and configurations, which can be obtained by, for example, arbitrarily combining the embodiments.

Although in the embodiment described above, there is adopted the configuration in which the hand part 24 is provided with the first hand part 241 and the second hand part 242, it is also possible for the hand part 24 to, for example, be configured only by the first hand part 241 without disposing the second hand part 242.

Although there is illustrated the configuration in which the first hand part 241, the second hand part 242, and the connection part 243 are each detachably attached, this configuration is not a limitation. For example, the firsthand part 241, the second hand part 242, and the connection part 243 can integrally be configured with each other. Specifically, a part of a shaft member having an elongated cylindrical shape is formed of a bellows part or the like, and the housing side of the bellows part is defined as the first hand part, and the opposite side (the control device side) is defined as the second hand part across the bellows part. In this case, it becomes possible to change the angle between the first hand part and the second hand part to an arbitrary angle in the bellows part.

Although in the embodiment described above, there is illustrated a configuration in which the cable 3 is inserted in the center axis of the hand part 24, the configuration is not a limitation.

For example, it is also possible to adopt a configuration in which an insertion opening for the cable 3 is disposed in a part of the housing, and the cable 3 is drawn from the insertion opening, and so on. In this case, the insertion opening is preferably disposed in the side surface 21C of the housing 21 similarly to the hand part 24.

Although there is shown the example in which the hand part 24 (the first hand part 241) has an axial direction parallel to the sensor surface 21A, this example is not a limitation.

For example, it is also possible for the first hand part 241 to be disposed at an angle with which the first hand part 241 crosses the sensor surface 21A. Further, it is also possible to adopt a configuration in which the installation angle of the first hand part 241 to the side surface 21C can arbitrarily be changed.

Although there is shown the example in which the image forming unit 142 processes the image generated based on the ultrasonic received signal using the rotation process, the horizontally flipping process, the vertically flipping process, and so on based on the attitude detection signal, and then makes the display section 12 display the image thus processed, it is also possible to adopt a configuration in which the image to be the reference can be designated when processing the image. For example, there is performed the image process so that such an image as shown in FIGS. 7A and 7B is displayed as the internal tomographic image with respect to the ultrasonic scan plane A shown in each of FIG. 7A, FIG. 8A, FIG. 9A, FIG. 10A, FIG. 11A, FIG. 12A, FIG. 13A, and FIG. 14A in the embodiment described above based on the arrangement of the ultrasonic probe 2 shown in FIG. 7A, even in the case in which the attitude of the ultrasonic probe 2 is changed. In contrast, it is also possible to adopt a configuration in which it is possible to set so that the image, which is generated based on the ultrasonic received signal when the hand part 24 is disposed on the back side as shown in FIGS. 11A through 11C, becomes the reference.

Further, it is also possible to adopt a configuration in which, for example, the image processing by the image forming unit 142 is not performed, and the image corresponding to the installation direction of the ultrasonic probe 2 is displayed on the display section 12.

Although there is shown the example in which the ultrasonic transducer array 50 having the one-dimensional array structure is disposed as the ultrasonic sensor 22, it is also possible to adopt a configuration in which, for example, an ultrasonic transducer array having a two-dimensional array structure is disposed. In this case, it is sufficient to draw the lower part electrodes 414 in the respective ultrasonic transducers 51 to the terminal region Ar2 independently of each other to constitute the configuration in which signals different from each other can be input to the respective ultrasonic transducers 51. By using such an ultrasonic transducer having the two-dimensional array structure, a three-dimensional image in the living body can also be obtained. Further, even in the case of such a two-dimensional array structure, by disposing the hand part 24 as shown in the embodiment described above, and changing the attitude of the ultrasonic probe using substantially the same operation, the three-dimensional images from a variety of angles can easily be obtained.

Although in the embodiment described above, there is illustrated the configuration in which the acoustic matching layer 43 and the acoustic lens 44 are disposed on the opening part 411A side as shown in FIG. 5, this configuration is not a limitation. It is also possible to adopt a configuration in which, for example, the opening parts 411A are located on the sealing plate 42 side (the reverse 41A side) , and the piezoelectric elements 413 are disposed on an operation surface 41B side of the element substrate 41. In this case, the acoustic matching layer 43 is formed on the operation surface 41B side of the element substrate 41 so as to cover the piezoelectric elements 413, and the acoustic lens 44 is formed on the surface of the acoustic matching layer 43.

Further, although there is shown the example in which the ultrasonic transducers 51 are each formed of the support film 412 for blocking the opening part 411A and the piezoelectric element 413 disposed on the support film 412, the example is not a limitation. It is also possible to adopt a configuration provided with, for example, a first electrode disposed on the substrate, a vibrating film disposed via a gap with respect to the substrate, and a second electrode disposed on the vibrating film and opposed to the first electrode. In the present configuration, a rectangular pulse wave is applied between the first electrode and the second electrode to thereby generate electrostatic attractive force between the electrodes to vibrate the vibrating film.

Although in the embodiment described above, there is shown the example in which the thickness dimension of the housing 21 is equal to or smaller than 1 cm, it is also possible to, for example, have a thickness dimension larger than 1 cm. In the case in which no other parts exist in the periphery, such as the case of performing the operation of an arm or a leg, the thickness dimension of the housing 21 can be made larger.

Further, although in the present embodiment, there is shown the example of using the part in the living body as the measurement object, this example is not a limitation, but the invention can also be implemented as an ultrasonic apparatus when measuring the internal structure in, for example, a concrete building. Even in such a case, it is possible to insert the ultrasonic probe 2 in a gap or the like of the structure, or to make the ultrasonic probe 2 have contact with a position which the hand of the operator does not reach in high places or the like using the hand part 24. In such a case, it is sufficient to set the thickness dimension of the housing in accordance with the structure size and so on. Further, in the case of using such a structure as the object, it is not required to form the side surface 21C of the housing 21 to have the convexly curved surface, and it is also possible that, for example, the side surface 21C perpendicular to the sensor surface 21A is disposed.

Although there is shown the example in which the second hand part 242 is formed of the plurality of partial hand parts 242A as shown in FIG. 6 as a configuration example of the second hand part 242, this configuration is not a limitation as described above. It is also possible to adopt a configuration in which, for example, the second hand part 242 can be attached and detached by moving the second hand part 242 along the cable 3 in the axial direction.

Further although in the first hand part 241, it is assumed that the first hand part 241 can be attached to and detached from the housing 21 by engaging the engaging claw 241A with the locking part 21F of the housing 21, this configuration is not a limitation. It is also possible to adopt a configuration in which, for example, the first hand part 241 can be attached to and detached from the housing 21 by screwing an external screw part disposed on the tip of the first hand part 241 to an internal screw part provided to the housing 21.

Such a variety of configuration examples as described above can also be used as an attaching/detaching configuration of the first hand part 241 and the connection part 243 and an attaching/detaching configuration of the second hand part 242 and the connection part 243. It is also possible to adopt a configuration of, for example, screwing an external screw part disposed at the tip of the first hand part 241 and the second hand part 242 to an internal screw part provided to the connection part 243, or it is also possible to adopt a configuration of engaging an engaging claw provided to the first hand part 241 and the second hand part 242 with an engaging groove provided to the connection part 243.

Besides the above, specific structures to be adopted when implementing the invention can be configured by arbitrarily combining the embodiments and the modified examples described above, or can arbitrarily be replaced with other structures and so on within the range in which the advantages of the invention can be achieved.

The entire disclosure of Japanese Patent Application No. 2015-149249 filed on Jul. 29, 2015 is expressly incorporated by reference herein.

What is claimed is:

1. An ultrasonic probe comprising:
a housing shaped like a thin plate and having a transmitting/receiving surface for an ultrasonic wave; and
a hand part having an axial direction, and connected to a side surface of the housing at one end in the axial direction, the side surface crossing the transmitting/receiving surface of the housing.

2. The ultrasonic probe according to claim 1, wherein
the hand part extends in a direction parallel to a surface direction of the transmitting/receiving surface.

3. The ultrasonic probe according to claim 1, wherein
the hand part is disposed so as to detachably be attached to the housing.

4. The ultrasonic probe according to claim 1, wherein
the hand part is provided with a first hand part connected to the housing, and a second hand part connected to the first hand part so that an angle with the first hand part can be changed.

5. The ultrasonic probe according to claim 1, wherein
the hand part is provided with a cylindrical inner peripheral surface along the axial direction, and
a cable to be connected to an ultrasonic sensor disposed inside the housing is disposed in a hollow part formed of the cylindrical inner peripheral surface.

6. The ultrasonic probe according to claim 1, wherein
the side surface of the housing is a convexly curved surface.

7. The ultrasonic probe according to claim 1, wherein
a thickness dimension along a normal line of the transmitting/receiving surface of the housing is no larger than 1 cm.

8. The ultrasonic probe according to claim 1, further comprising:
a plurality of ultrasonic transducers each including a support film and a vibrator shaped like a film and adapted to vibrate the support film,
wherein the ultrasonic transducers are arranged in an array along a surface direction of the transmitting/receiving surface.

9. An ultrasonic apparatus comprising:
the ultrasonic probe according to claim 1; and
an image forming section adapted to form an image based on a signal output from the ultrasonic probe.

10. The ultrasonic apparatus according to claim 9, further comprising:
an attitude detection section adapted to detect an attitude of the ultrasonic probe,
wherein the image forming section one of rotates and flips the image in accordance with the attitude of the ultrasonic probe.
